(19) United States
(12) Patent Application Publication
(10) Pub. No.: US 2025/0064896 A1
(43) Pub. Date: Feb. 27, 2025

(54) METHODS, COMPOSITIONS AND USES FOR TREATING CANCER BY PROVIDING MEDICATIONS THAT INDUCE TARGETED TUMOR CELL MITOSIS BEFORE PROVIDING CHEMOTHERAPY OR RADIATION AND KITS THEREFOR

(71) Applicant: MAISON AMORI OQVPO LLC, New York, NY (US)

(72) Inventor: Nicola J. NASSER, New York, NY (US)

(21) Appl. No.: 18/794,331

(22) Filed: Aug. 5, 2024

Related U.S. Application Data
(63) Continuation of application No. PCT/US2023/012275, filed on Feb. 3, 2023.
(60) Provisional application No. 63/306,123, filed on Feb. 3, 2022.

(51) Int. Cl. A61K 38/24; A61K 31/4178; A61K 31/566

(52) U.S. Cl. CPC A61K 38/24; A61K 31/4178; A61K 31/566

(57) ABSTRACT

Methods, compositions, uses and kits can be provided to treat cancer in a subject, which can include administering to the subject an active agent to induce enhanced tumor cell mitosis, and providing, in proximity or immediately after that, a therapy that specifically targets cells in mitosis, such as chemotherapy or radiation.

### CROSS REFERENCE TO RELATED APPLICATION(S)

This application relates to and claims priority from U.S. Patent Application No. 63/306,123, filed Feb. 3, 2022, the entire disclosure of which is incorporated herein by reference.

### FIELD OF THE DISCLOSURE

An exemplary embodiment of the present disclosure relates generally to a methods to treat various cancers, and more specifically to such methods for treating cancers that have specific molecules and/or receptors that result in cell division once activated, by providing an agonist to those receptors in a controlled manner, to induce enhanced tumor cell mitosis, and to provide in proximity or immediately after that, therapies that specifically target cells in mitosis, such as chemotherapy or radiation. Another exemplary embodiment of the present disclosure relates to a method comprising: administering an immediate release formulation of luteinizing hormone (LH) Releasing Hormone (RH) to induce a testosterone surge, in a subject, such as a man, suffering from impotence due to low androgen levels.

### BACKGROUND INFORMATION

In 1941, Charles Huggins and Clarence V. Hodges, published a series of 8 patients with carcinoma of the prostate metastatic to bone who underwent bilateral orchiectomy. Since then, castration became the main modality of treatment of metastatic prostate cancer. The first evidence for the existence of hypothalamic substances that controlled the secretion of hormones from the anterior pituitary gland was reported by Saffran and Schally in 1955 when they identified the corticotropin-releasing factor (CRF). The purification and identification of the LHRH sequence of 10 amino acids was reported by the group of Schally in 1971. The understanding of the mechanism of action of LHRH, and the development of long-acting agonists and antagonists to LHRH, resulted in a shift from surgical castration to the use of long acting LHRH agonists and antagonists to induce castrate androgen levels. LHRH causes the pituitary gland to produce luteinizing hormone which results in synthesis and secretion of testosterone from the testicles. When LHRH levels are continuously high, the pituitary gland stops producing LH, which results in reduced testosterone production by the testicles.

The seminal study by Bolla et al that compared external irradiation with external irradiation plus goserelin, an agonist analogue of gonadotropin-releasing hormone, showed survival benefit for adding hormonal therapy to radiation compared to radiation alone. Warde et al. randomized patients with locally advanced prostate cancer to androgen deprivation therapy (ADT), versus radiation therapy and ADT (LHRH agonist used in 92% and orchiectomy in 8% of the patients), and showed that the combined therapy resulted in a better survival. Thus, combining LHRH agonist with radiation for locally advanced prostate cancer results in a survival benefit, compared to either of the treatments alone. This means that there is more tumor cell kill when LHRH agonist is combined with radiation.

Publication by Nasser et al. has shown (Nasser NJ, Cancer 2022) that the beneficial part of combining LHRH and radiation is the androgen flare and not androgen deprivation, and that androgen suppression could result in resistance to radiation and in the need for dose escalation.

LHRH agonists results in androgen flare before ADT is achieved: Long acting LHRH agonists result in a surge in androgens which starts a few hours after its administration, and lasts for a few days. The HERO study by Shore et al. randomly assigned patients with advanced prostate cancer to daily oral relugolix (LHRH antagonist) or long-acting leuprolide (LHRH long-acting agonist) injections once every 3 months. The authors measured testosterone levels as a function of time from the start of LHRH agonist. At week 2 from the initiation of leuprolide, testosterone levels were about 50% higher compared to baseline. Klotz et al. published a study that evaluated the efficacy and safety of degarelix (LHRH antagonist) versus monthly leuprolide. After the initiation of leuprolide, median testosterone levels increased by 65% from baseline by day 3 (median testosterone level 6.30 ng/ml; P<0.001). Median testosterone levels remained above castrate levels (0.5 ng/ml) until day 21. Sasagawa et al. measured serum concentrations of LH and testosterone in 16 patients with advanced prostatic cancer before and after treatment with leuprolide. The increase in relative LH values was noted for 7 days with a maximum of 345±108% (means±standard error) on day 2 after LHRH analogue injection. For testosterone, elevation of its levels after LHRH analogue application was noted for 7 days, with a maximum of 145±13% on day 2. Thus, there is a period of about 7-15 days of testosterone flare after the initiation of LHRH agonist, which is much more extended in some patients, and likely depends on the LHRH type and its dose.

Antiandrogens do not result in castration, and there is no high level of evidence to show that it protects from testosterone flare. Bicalutamide, an antiandrogen, when used as monotherapy for patients with prostate cancer results in a rise in LH, oestradiol, and testosterone levels. Nonsteroidal antiandrogens are regarded as a therapeutic option for patients with advanced prostate cancer who wish to retain sexual interest and function. Early Prostate Cancer trial showed that the patients randomized to bicalutamide had a non-statistically significant difference in erectile dysfunction compared to patients treated with placebo. Prostate-specific antigen decreases under treatment with bicalutamide, and that's why it is erroneously called a “castrating” medication, despite that most potent patients maintain potency after the initiation of antiandrogen monotherapy.

In patients with a surge in testosterone, as occurs during the first 1-2 weeks of LHRH therapy, the benefit of antiandrogen is much more obscure. The thought that anti-androgen can “protect” from testosterone flare was investigated in multiple studies. Oh et al. identified newly diagnosed metastatic prostate cancer patients treated at the Veterans Affairs Hospitals from 2001-2004 with LHRH agonists with or without prior anti-androgen therapy. Antiandrogen therapy before LHRH agonist initiation in patients with metastatic prostate cancer was not associated with differences in fractures, spinal cord compression, bladder outlet obstruction, or change in narcotic prescription. Vis et al. reviewed the literature regarding testosterone flare, and found that there is a lack of compelling data showing definite disease progression during the short period of testosterone flare after initiation of LHRH agonist therapy. A more recent review of the literature by Krakowsky et al. found that testosterone flare does not appear to be associated with significantly increased PSA, disease progression, or adverse events, even in men with widely metastatic disease. Testosterone flare after initiation of LHRH could theoretically result in symptomatic progression of prostate cancer, and thus the role of antiandrogens in preventing these side effects is controversial.

Radiation with bicalutamide therapy in patients with recurrent prostate cancer, showed significantly higher rates of long-term overall survival compared to radiation alone. ADT in the salvage setting showed survival benefit only when goserelin was provided on the first day of radiation, as in GETUG-AFU 16. Thus, starting LHRH therapy before radiation is not supported by any randomized trial in the salvage setting. Neoadjuvant ADT shouldn't be a substitute for bicalutamide, or provided instead of starting LHRH on day 1 or radiation.

Androgens drive prostate cancer cells into mitosis: The androgen receptor pathway is a key driver of prostate cancer progression. Androgen activates the androgen receptor which is critical for survival and proliferation of androgen-sensitive prostate cancer cells. The seminal studies by Huggins et al., and multiple studies published after that, highlighted androgen deprivation as pivotal in the management of advanced prostate cancer and high-risk localized disease. Androgen deprivation has significant side effects: impotence, hypertension, obesity, and diabetes. Thus, there is a need for medications capable of curing prostate cancer without androgen deprivation. Docetaxel is a chemotherapy that is effective against prostate cancer, and specifically targets cells during cell division. By stabilizing the mitotic spindle, docetaxel induces “mitotic catastrophe” and death of the dividing cancer cells. Docetaxel when given at the beginning of LHRH therapy for patients with metastatic hormone sensitive prostate cancer (MHSPC) results in significantly longer overall survival than LHRH therapy alone. Nasser et al. recently published (Cancers 2022) a secondary analysis of the CHAARTED trial which randomized MHSPC patients to ADT alone or ADT plus docetaxel. The study showed that by providing the first dose of docetaxel during testosterone flare, at 1-6 days from LHRH initiation, patients could have better clinical outcomes, compared to patients who started docetaxel more than 14 days from LHRH initiation, as testosterone drives specifically prostate cells into mitosis, priming it to cell kill by docetaxel. Overall survival was 72 versus 57 months (p=0.2) for patients who started docetaxel 1-6 days versus >14 days from initiation of LHRH agonist, respectively. Median actuarial freedom from Castrate Resistant Prostate Cancer (CRPC) was 51 versus 18 months (p=0.04) for patients who started docetaxel 1-6 days compared to >14 days from LHRH initiation, respectively.

Kishan et al. performed a systemic meta-analysis of clinical trials that evaluated the use or prolongation of androgen deprivation therapy (ADT) (or both) in men with localized prostate cancer receiving definitive radiotherapy. The authors found that adding ADT during radiation therapy and prolonging the portion of ADT that follows radiotherapy is associated with improved metastasis-free survival in men. The authors did not note that the ADT provided for most patients was LHRH agonist, that induces testosterone surge upon its initiation.

Mitotic death is the dominant mechanism of cancer cell kill following radiation: For most cells, death while attempting to divide, that is, mitotic death, is the dominant mechanism cell kill following radiation. Radiation therapy, similar to treatment with docetaxel, targets mainly dividing cancer cells in mitosis. Combining LHRH and radiation therapy was shown to increase survival of prostate cancer patients with locally advanced disease, and in patients with a low metastatic burden. The mechanism of synergism between LHRH and radiation is not clear. Our preliminary evidence based on previous randomized controlled trials, shows that androgen surge during radiation, rather than its suppression, could be responsible for the enhanced prostate cancer cell kill (Nasser NJ, Cancers 2022).

The linear-quadratic model is used in radiation oncology to estimate tumor control probability and normal tissue complication probability using logistic models. The alpha/beta ratio is the dose where the linear and the quadratic component causes the same amount of cell kill. Rapidly proliferating tumors, such as lymphoma and non-small cell lung cancer, have a high alpha/beta ratio of 10 Gy or more. Prostate cancer has a lower alpha/beta ratio of about 1.5-3 Gy. These alpha/beta ratios of the prostate were measured under radiation therapy alone, or in combination with ADT. During androgen flare, the number of mitotic cancer cells increases, and thus the alpha/beta ratio at that time will necessarily be higher than that without androgen stimulation or during androgen deprivation. How high alpha/beta ratios of prostate go during androgen flare is a matter that will need to be investigated radiobiologically, but it probably could reach 10 Gy or even more, as radiation therapy spanning a short period of testosterone flare, as in Radiation Therapy Oncology Group (RTOG) 8531 trial and in European Organization for Research and Treatment of Cancer (EORTC) 22863 study resulted in significant absolute survival benefits, compared to radiation only, of about 10-20% at 10 years of follow-up. This as opposed to the RTOG 9413 trial in which radiation during testosterone flare was avoided and providing ADT before or after radiation did not result in any significant difference in survival.

Studies testing the combination of LHRH agonist and radiation: RTOG 8531 trial was a national prospective randomized trial of standard external-beam irradiation, plus the LHRH agonist, goserelin, which was started in the last week of radiation and delivered indefinitely or until the sign of disease progression (arm I), versus radiation alone with hormone manipulation at the time of relapse (arm II). The initial target volume was the whole pelvis, and was treated with 45 Gy. The prostatic boost volume received 20 to 25 Gy, bringing the total prescribed dose to that volume to 65-70 Gy. The 5-and 9-year absolute survival rates were 72% and 62%, respectively, for all patients in arm I, and 50% and 38%, respectively, for all patients in arm II. P value was 0.23 on univariate analysis, but on multivariate analysis results were statistically significant (P=0.030). Thus at 5 and 9 years there was respectively 22% and 24% absolute difference in overall survival between the two arms. At 10 years, the absolute survival rate was significantly greater for arm 1 than for the control arm: 49% vs. 39%, respectively (p=0.002).

The EORTC 22863 study by Bolla et al was a randomized, prospective trial comparing external irradiation with external irradiation plus goserelin, which was started on the first day of irradiation and continued for 3 years, and an antiandrogen that was given for 1 month starting a week before the first goserelin injection. Five-year overall survival was 62% and 78%, respectively (p=0.0002), 10-year overall survival was 39.8% and 58.1% (p=0.0004), in patients receiving radiotherapy alone compared to those allocated to combined treatment, an absolute overall survival difference between the two arms of 16% and 18.3%, at 5 and 10 years, respectively. A subsequent study from the EORTC investigated the benefit of prolonged hormonal therapy after radiation and hormonal therapy in patients with locally advanced prostate cancer. All patients received external-beam radiotherapy plus 6 months of LHRH analogue initiated on the first day of radiation, and an antiandrogen agent (flutamide or bicalutamide), initiated 1 week before the start of treatment with the LHRH analogue. After completing 6 months of hormonal therapy the patients were randomized to observation or treatment with the same LHRH analogue but without the antiandrogen for another 2.5 years. The 5-year over-all survival was 81% and 84.8% for the short-and long-term hormonal therapy, respectively, a 3.8% survival benefit for the additional 2.5 years of hormonal therapy. So, this study together with EORTC 22863 shows that the main survival benefit for combined therapy is the radiation and the first 6 months of hormonal therapy in which LHRH was provided starting on the first day of radiation.

RTOG 8610 was the first phase III randomized trial to evaluate neoadjuvant ADT in combination with external-beam radiotherapy in men with locally advanced prostate cancer. Patients received combined ADT that consisted of goserelin 3.6 mg every 4 weeks and flutamide 250 mg tid for 2 months before and concurrent with RT, or they received RT alone. There was no significant difference in survival between the two groups.

RTOG 9202 was a phase 3 trial that randomized 1,554 patients with locally advanced prostate cancer with PSA<150 ng/ml, who completed 4 months of goserelin and flutamide, 2 months before and 2 months during RT to a dose of 65 to 70 Gy to the prostate and 44 to 50 Gy to the pelvic lymph nodes, to 24 months of goserelin or no further treatment. Overall survival was not significantly different between the two treatment arms; 80.0% v 78.5% at 5 years, P=0.73.

RTOG 9413 was a 2×2 factorial study, that tried to prove that better castration at the start of radiation therapy could result in survival benefit compared to providing hormonal therapy after radiation, and that radiation to the whole pelvis is superior to prostate only in patients with a locally advanced prostate cancer. All patients received LHRH agonist, goserelin or leuprolide, and an antiandrogen, flutamide, for 4 months. First group began hormonal therapy 2 months before radiation and continued to receive it during radiation, whereas the other group began hormonal therapy immediately following the completion of radiation. Radiation therapy (RT) was given at 1.8 Gy/fraction to a total dose of 70.2 Gy. Whole Pelvis (WP) RT consisted of conventional four-field “box” technique with a minimum unblocked field size of 16×16 cm to a dose of 50.4 Gy, followed by an additional 19.8 Gy to the prostate. Prostate-only (PO) RT was limited to the prostate and seminal vesicles, with a maximum unblocked field size of 11×11 cm to a total dose of 70.2 Gy. The 10-year estimates of overall survival did not differ significantly between the groups. This led the authors to conclude that there are sequence-dependent and volume-dependent interactions between hormonal therapy and radiotherapy. Without being bound by theory, the exemplary embodiments of the present disclosure can provide an indication of the potential interaction between the sequence of hormonal therapy and the volume of the radiation fields in the treatment of prostate cancer.

Testosterone flare after luteinizing hormone-releasing hormone injection is the side effect that makes most of the beneficial effect when it coincides with radiation therapy for prostate cancer: While radiation therapy to a dose of 70 Gy could be sufficient to kill non-mitotic prostate cancer cells, 45-50 Gy could be sufficient to kill mitotic prostate cancer cells. Providing neoadjuvant hormonal therapy, as in RTOG 9413, results in driving tumor cells into mitosis during the androgen flare, followed by suppression of tumor cell mitosis when androgen deprivation is achieved, and when radiation is delivered. Thus, the radiation in this arm was delivered while the cells are resistant to radiation. This is opposed to RTOG 8531 and EORTC 22863 when part of the radiation is delivered during androgen flare that drives prostate cancer cells into mitosis, priming them to cell kill by radiation. Neoadjuvant hormonal therapy failed to provide survival benefit. Recent metanalysis showed that longer extension of total ADT duration in the neoadjuvant setting from 3-4 months to 6-9 months did not result in survival benefit, and there was no survival benefit for adjuvant short term ADT versus long term ADT at 10 years (66% versus 67%). This as opposed to EORTC study that showed modest survival benefit for extending adjuvant ADT from 6 month to 3 years. Thus, the main survival benefit in combining hormonal therapy and radiation is achieved from starting LHRH during radiation, not before or after. Testosterone flare is the most reasonable explanation for the enhanced sensitivity of prostate cancer cells when radiation therapy is combined with LHRH.

Adjei et al. tested the use of intranasal leuprolide. Though instead of focusing on its effects on androgen levels, this group focused on leuprolide blood levels, which was as expected much lower than the intravenous route. The delivery of LHRH is intended to reach the pituitary, and systemic effects of the releasing hormone per se are not known, thus the most important data, is the effect on testosterone levels. The fact that leuprolide reaches the blood when delivered intranasally strengthen the possibility that it could reach the pituitary with enough levels to trigger secretion of LH, which could result in secretion of testosterone from the testicles.

Publication by Fowler Jr, J. E., & Whitmore, published in 1982, provides a report about a series of patients with prostate cancer treated with testosterone to induce tumor progression, followed by chemotherapy. Testosterone was delivered daily, with a mean and median duration of treatment of 42.4 and 9 days, respectively. Among 52 patients with metastatic adenocarcinoma of the prostate who were treated with exogenous testosterone, 45 (87%) experienced unfavorable subjective and/or objective responses. Serious morbidity or mortality, seemingly due to the testosterone administration, occurred in eight cases (15%). The main drawback of this study is that it provided testosterone which has a half-life of 7 days, more than 1-7 weeks. Thus, high testosterone induced tumor cell mitosis way more time than chemotherapy was in the patients' blood. The chemotherapy that was used was not reported, and at that time, docetaxel, the chemotherapy that is known to have activity against prostate cancer was not in use. Furthermore, androgen flare after LHRH administration may include the release of androgens other than testosterone that are more effective in priming prostate cancer cells to docetaxel.

Accordingly, there may be a need to address and/or at least partially overcome at least some of the prior deficiencies described herein.

### SUMMARY OF EXEMPLARY EMBODIMENTS

To at least partially address and/or overcome such issues and/or deficiencies, methods, compositions, and uses thereof for treating cancer, such as, e.g., prostate cancer, can be provided according to certain exemplary embodiments of the present disclosure.

According to certain exemplary embodiment of the present disclosure, a method for treating cancer can be provided. The exemplary methods can include, for example, administering a first molecule that results in cancer cell mitosis and/or progression, followed subsequently by administering a second molecule or treatment that specifically targets dividing cells.

According to certain exemplary embodiment of the present disclosure, the cancer can be prostate cancer, and the molecule that induce specifically cancer cell mitosis can be immediately acting LHRH (e.g. Gonadorelin) or testosterone.

According to certain exemplary embodiment of the present disclosure, the cancer can be breast cancer, and the molecule that induce specifically cancer cell mitosis can be immediately acting LHRH or estrogen or progesterone.

According to certain exemplary embodiment of the present disclosure, molecules or treatment that specifically target dividing cells can include, but are not necessarily limited to, chemotherapy using, for example, at least one of an anthracycline, a plant Alkaloid, a taxane, a vinca alkaloid; a platinum-based chemotherapy, an antimetabolite, or a topoisomerase Inhibitor, or a combination thereof; or radiation.

The mechanism of action in the methods according to certain exemplary embodiment of the present disclosure can be increasing tumor cell mitosis, with an antimitotic treatment delivered immediately after that and acting once the tumor cells in mitosis, and resulting in increased tumor cell kill.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary schematic drawing showing the pharmacokinetics of androgens after long acting LHRH initiation;

FIG. 1B is an exemplary schematic drawing showing that if long acting LHRH is delivered alone, there is initial increase in prostate cancer cell count before stabilization occurs;

FIG. 1C is an exemplary schematic drawing showing that if radiation therapy is started 6-8 weeks after long acting LHRH initiation, there will be more resistance, necessitating radiation dose escalation. Kishan et al. performed a systemic meta-analysis of clinical trials that evaluated the use or prolongation of androgen deprivation therapy (ADT) and found that adding ADT during radiation therapy and prolonging the portion of ADT that follows radiotherapy is associated with improved metastasis-free survival in men, but starting and prolonging ADT before radiation was not associated with improved outcomes;

FIG. 1D is an exemplary schematic drawing showing that the best timing of combining long acting LHRH initiation and radiation may be delivering LHRH on the first day of radiation as done by Bolla et al. ensuring that the patient got radiation treatment during androgen flare;

FIGS. 2A and 2B show exemplary differences in androgen levels after the administration of Long-acting continuous release LHRH 4 with activity over about 3 months (see FIG. 2A), as compared to repeated injections of short acting immediate release LHRH 21 (see FIG. 2B);

FIG. 3 illustrates an exemplary method for administering LHRH or an LHRH agonist through the intranasal route, so that some of it, passes the blood brain barrier and reaches the pituitary gland, resulting in secretion of LH from the pituitary;

FIG. 4 shows typical radiation fields used during RTOG 9413. Doses of radiation of 45-50.4 Gy may not be enough to kill dormant tumor cells, but these doses could be cytotoxic to tumor cells in mitosis. Doses of 65-70.2 Gy were used previously and higher doses as high as 86.4 Gy to target the prostate on patients under androgen deprivation are used. Patients on continuous androgen flare may need much lower doses of radiation;

FIG. 5 is a schematic drawing showing an exemplary method comprising releasing medication in a cyclic manner (e.g., intermittent release) by encapsulating it in a plurality of capsules with different thicknesses;

FIG. 6 is a schematic drawing showing an exemplary method comprising releasing medication in a cyclic manner by encapsulating it in plurality of capsules with different material densities; and

FIG. 7 is a schematic drawing showing an exemplary method comprising releasing medication in a cyclic manner by encapsulating it in capsules with different thicknesses, using as well master capsules to extend the times that the medication is secreted in the cyclic manner.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary embodiments of the present disclosure are described and illustrated herein by the following examples, which should not be construed as limiting. The contents of all references, pending patent applications and published patents, cited throughout this application are hereby expressly incorporated by reference. Those skilled in the art will understand that these exemplary embodiments of the present disclosure may be embodied in many different forms and should not be construed as limited to the embodiments set forth herein. Rather, these exemplary embodiments are provided so that the present disclosure will fully convey the exemplary embodiment of the present disclosure to those skilled in the art. Various exemplary modifications and other exemplary embodiments of the present disclosure will come to mind in one skilled in the art to which this invention pertains having the benefit of the teachings presented in the foregoing description. Although specific terms are employed, they are used as in the art unless otherwise indicated.

According to certain exemplary embodiments of the present disclosure, the following methods, compositions, products, kits and uses thereof can be provided:
[1]. A method for treating cancer in a subject, comprising:
(a) administering to the subject a medication in an amount effective to specifically induces mitosis of cancer cells, and
(b) applying a treatment that kills cells in mitosis, such as a chemotherapy or a radiation therapy, 8-150 hours, 12-150 hours, or 8-120 hours after (1).
[2]. The method according to [1], wherein the cancer is prostate cancer, and wherein the method comprises:
   administering to the subject an immediate release LHRH agonist, such as Gonadorelin or a short acting Leuprolide, to induce an androgen surge without an ensuing androgen deprivation so that cells having an androgen receptor proceed to mitosis, and.
   administering to the subject, 8-120 hours after administering the immediate release LHRH agonist, a treatment that kills cells in mitosis, such as a chemotherapy (e.g. docetaxel) or a radiation therapy.
[3]. The method according to [1], wherein the cancer is hormone receptor positive breast cancer, wherein the subject is a premenopausal woman, and wherein the method comprises:
   administering to the subject an LHRH agonist, such as subcutaneous leuprolide, at a dose of 0.1-1 mg/d starting day 21 of the menstrual cycle, to suppress the effect of the pituitary axis,
   administering to the subject, at day 7 of the next cycle, intravenous estrogen (0.001-5 mg) to estrogen receptor positive, or administering intravenous progesterone to progesterone receptor positive breast cancer, to specifically increase the part of tumor cells in mitosis, followed by chemotherapy or radiation 8-120 hours after that.
[4]. The method according to [1], wherein the cancer is hormone receptor positive breast cancer, wherein the subject is a premenopausal woman, and wherein the method comprises:
   Administering to the subject LHRH agonist such as subcutaneous leuprolide at a dose of 0.1-1 mg/d starting day 21 of a menstrual cycle, to suppress the effect of the pituitary axis,
   at day 1 of the next menstrual cycle, starting injecting follicle stimulating hormone (FSH) injections once daily for 8 days, at days 8-14, administering human chorionic gonadotropin (HCG) or LH, and at days 8-16, administering chemotherapy for estrogen receptor positive breast cancer, or at days 12-20 administering chemotherapy for progesterone receptor positive breast cancer.
[5]. The method according to [4], wherein the chemotherapy is administered about 20-120 hours from a peak estrogen level or a peak progesterone level.
[6]. The method according to [1], wherein the cancer is a hormone receptor positive breast cancer, and wherein the method further comprises administering to the subject estrogen or progesterone, or an analogue thereof, in an amount effective to increase the portion of cancer cells in mitosis 8-120 hours before each cycle of the chemotherapy or the radiation therapy.
[7]. The method according to [1], wherein the cancer is human epidermal receptor 2 positive breast cancer, wherein the method further comprises administering a molecule, such as an antibody, that specifically activates human epidermal growth factor receptor 2 8-120 hours before applying the treatment in step (b).
[8]. The method according to [1], wherein the medication in step (a) comprises a hormone or an analogue of a hormone, such as testosterone, estrogen, progesterone, leuprolide, Gonadorelin, a Luteinizing Hormone-Releasing Hormone analogue, Corticotropin-releasing hormone, thyrotropin-releasing hormone, or Growth hormone-releasing hormone.
[9]. The method according to any one of [1]-[8], wherein the medications that induce tumor cell mitosis is administered intravenously, subcutaneously, transdermally, transbuccally, through an inhaler, by intranasal spray, or by a long intranasal applicator reaching as close as possible to the pituitary gland.
[10]. The method according to any one of [1]-[9], wherein LHRH is encapsulated in a plurality of capsules formulations that have different release times, so that formulation n+1 is released only after specific time from the release of LHRH from formulation n, resulting in a cyclic secretion of LHRH, with a peak between secretions ranging between 2 hours to 7 days, to achieve continuously higher than normal levels of testosterone.
[11]. A method for treating impotence in a subject with a low testosterone level, comprising:
   administering to the subject short acting, immediate release LHRH, such as Gonadorelin, or an analogue thereof, via a self-delivery, pen shaped, subcutaneous needle injection, intranasal spray, trans buccal formulation, or through inhalers once every 1-4 weeks, or.
   administering to the subject an LHRH composition as described in [9], once every 1-6 months.
[12]. The method according to any one of claims [1]-[11], wherein the chemotherapy includes using at least one of an anthracycline, a plant alkaloid, a taxane, a vinca alkaloid; a platinum-based chemotherapy, an antimetabolite, or a topoisomerase inhibitor, or a combination thereof.
[13]. A kit comprising:
   a self-injectable prefilled needle, that preferably pen-shaped, comprising a prefilled syringe that is permanent or disposable, that include an active agent comprising LHRH, an LHRH agonist, a decapeptide (pGlu-His-Trp-Ser-Tyr-D-Trp-Leu-Arg-Pro-Gly-NH2; SEQ ID NO:1), Gonadorelin, leuprolide, a modification thereof, or a combination thereof, and
   an instruction for administering to a subject said active agent 8-150 hours, or 8-120 hours before starting chemotherapy.
[14]. A method for treating a hormone sensitive malignancy, such as prostate cancer or breast cancer in a subject, the method comprising:
   administering to the subject an immediate release LHRH agonist or testosterone for treating prostate cancer 8-48 hours before the start of the radiation week so radiation is delivered when tumor cells are dividing; or
   administering estrogen or progesterone for treating breast cancer 8-48 hours before the start of the radiation week, so radiation is delivered when tumor cells are dividing.
[15]. A method for treating hormone receptor positive breast cancer, in a premenopausal subject, comprising:
   providing a mitosis-targeting chemotherapy when an ovary of the subject is in a follicular phase of a menstrual cycle and/or when estradiol approaches peak levels for estrogen receptor positive cancer; or
   providing a mitosis-targeting chemotherapy for estrogen receptor negative, progesterone receptor positive, breast cancer during a luteal phase of an ovarian cycle of the subject and/or when progesterone reaches a peak level.
[16]. A method according to any one of [1]-[15], wherein the chemotherapy is administered by a continuous infusion pump, and wherein the tumor-cell-kill by the chemotherapy spans the period in which the tumor cells are in mitosis, preferably 8-150 hours after the induction of tumor cell mitosis.
[17]. A method, comprising delivering a therapeutic agent targeting the pituitary gland, such as a medication, a peptide or a hormone, alone or together with a pharmaceutically acceptable carrier through an intranasal device, wherein the therapeutic agent include Gonadorelin, leuprolide, a Luteinizing Hormone-Releasing Hormone analogue, a Corticotropin-releasing hormone, a thyrotropin-releasing hormone, or a Growth hormone-releasing hormone.
[18]. The method according to [1], wherein the medication that specifically induces mitosis of cancer cells is gonadorelin, and wherein 10 mcg-100 meg gonadorelin is administered intravenously or subcutaneously 8-120 hours before the chemotherapy or the radiation therapy.
[19]. The method according to [17], wherein the therapeutic agent is delivered within a carrier that allows passing through bone, such as a lipophilic, liposomal carrier.

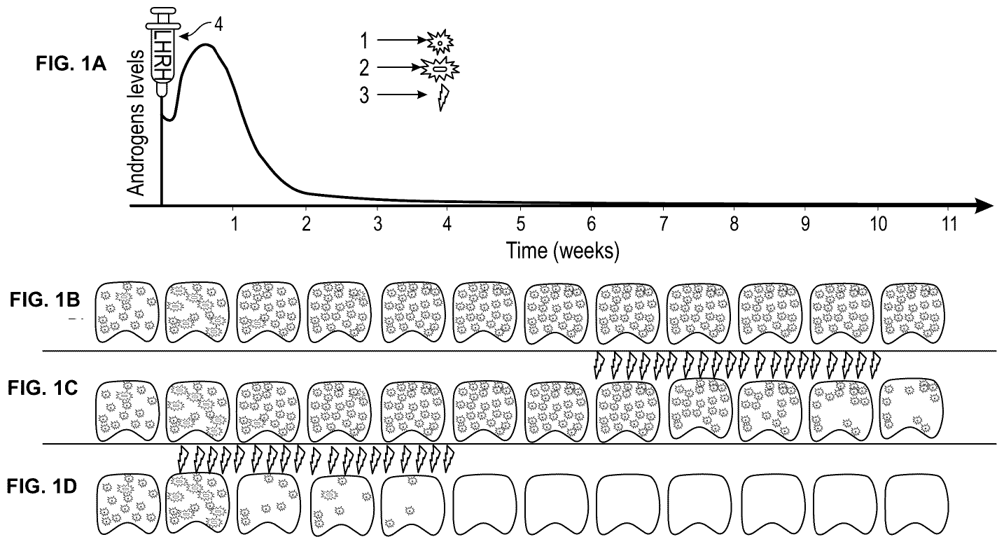

For example, FIGS. 1A-1D provided illustrations of cancer cells 1, cancer cell in mitosis 2, and Radiation 3. As illustrated in FIG. 1A, the pharmacokinetics of androgens after long-acting luteinizing hormone Releasing Hormone (LHRH) 4 injection are provided. After long acting LHRH injection 4 testosterone levels surges, increasing from baseline levels, before castrate levels are achieved. FIG. 1B shows what happens after long-acting luteinizing hormone Releasing Hormone (LHRH) 4 injection is provided alone, and indicates that there will be initial increase tumor cell division in the prostate before mitosis is suppressed because of castrate androgen levels. FIG. 1C shows what happen if long-acting luteinizing hormone Releasing Hormone (LHRH) 4 injection is combined with radiation therapy, when androgen levels reach castrate levels at 6-10 weeks, as done in most places in the United States, tumor cells kill still happens, but there is a need for dose escalation of the radiation. FIG. 1D shows what happens when radiation therapy is started in the same day of the first injection of long-acting luteinizing hormone Releasing Hormone (LHRH) 4; part of the radiation will be during androgen flare when specifically prostate cancer cells are in mitosis, priming these cells to cell kill by radiation.

Instead of providing long acting LHRH agonist, according to an exemplary method of an exemplary embodiment of the present disclosure, it is possible to administer a short acting, immediate release LHRH agonist, such as gonadorelin, that can result in an increase in androgen levels during radiation without inducing androgen deprivation. This can result in higher tumor cell kill, and can avoid impotence induced by long acting LHRH formulations. This can result in a possible need for a lower dose of radiation to cure prostate cancer.

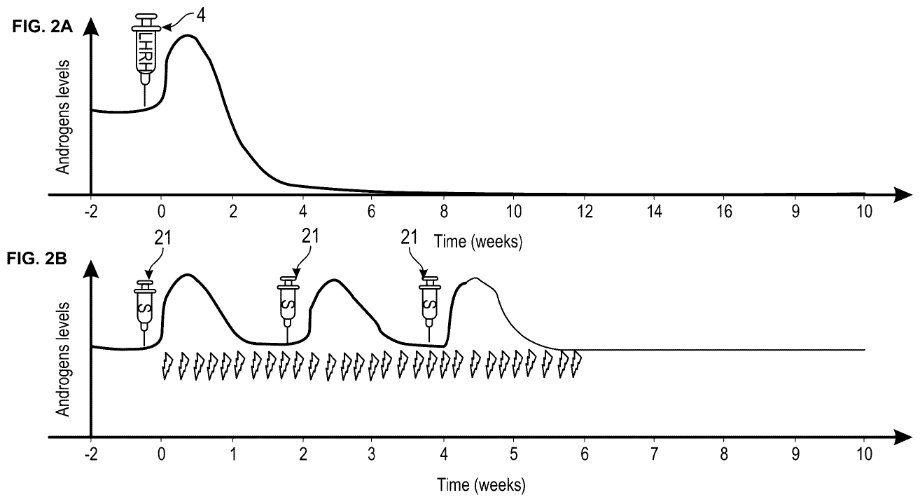

FIGS. 2A and 2B show exemplary graphs of the differences in androgen levels after the administration of Long-acting continuous release LHRH 4 with activity over about 3 months (see FIG. 2A), as compared to repeated injections of short acting immediate release LHRH 21 (see FIG. 2B) where testosterone levels never reach castrate levels. Administering radiation during testosterone flare can results in a higher cure rate, with the use of a lower dose of radiation.

According to certain exemplary embodiments of the present disclosure, exemplary methods can be provided, comprising administering LHRH, a LHRH analog or derivative, or an LHRH agonist through the intranasal route. Some of the LHRH, the LHRH analog or derivative, or the LHRH agonist administered may pass the blood brain barrier and reach the pituitary gland, resulting in secretion of LH from the pituitary. Adjei et al. Showed that leuproglide in the inhalation or intranasal route reaches the blood.

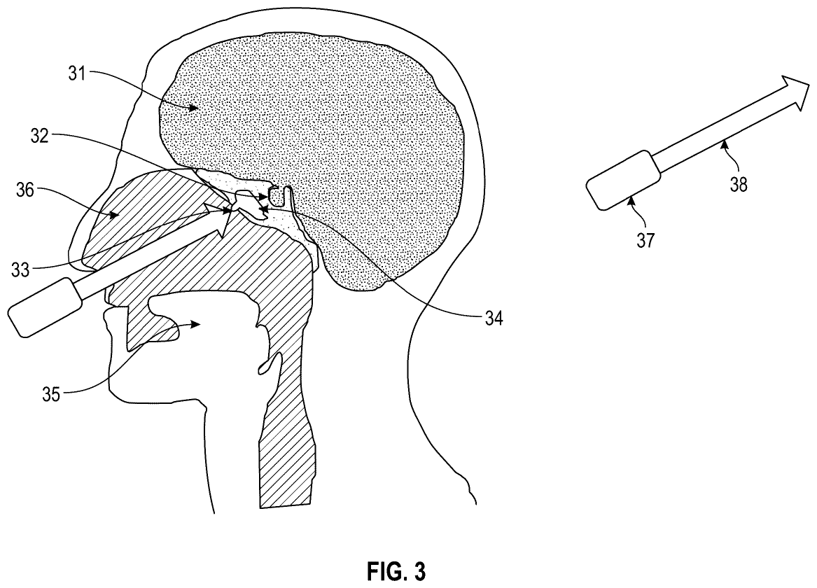

According to an exemplary embodiment of the present disclosure, as shown in FIG. 3A, a trans nasal pituitary spraying device can be provided which has a reservoir containing, for example, LHRH, an LHRH analog or derivative, an LHRH agonist or an other LH-releasing hormone or an analog or derivative thereof 37, within or without a liposomal formulation, that has long spraying arm that passes 38 through the nose 36 as close as possible to the opening of the sphenoid sinus 33 into the nasal cavity 36. This facilitates LHRH or other releasing hormones to reach to the brain 31, or pituitary gland 32 directly, or indirectly through absorption of the releasing hormone to the blood and from there through the blood brain barrier to the pituitary gland. Administering the immediate release LHRH through direct contact with the tongue 35, or oral mucosa can also result in avoiding injections and pain to the patients.

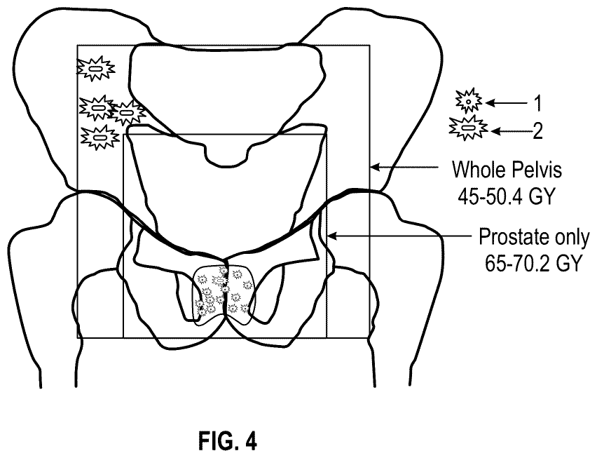

FIG. 4 shows an illustration of the radiation fields traditionally being used to treat prostate cancer. “1” denotes cancer cells. “2” denotes cancer cell in mitosis. Whole Pelvis field was usually treated with 45-50.4 Gy, and prostate is treated to 65-70.2 Gy. The use of radiation during androgen surge could results in specifically driving prostate cancer cells into mitosis, which make them more susceptible to cell kill by radiation, as radiation kills preferentially diving cells. Thus, if androgen surge is achieved through the whole course of radiation, then there may be a need for less radiation to cure prostate cancer, dose de-escalation, which could decrease the side effects of radiation to normal organs.

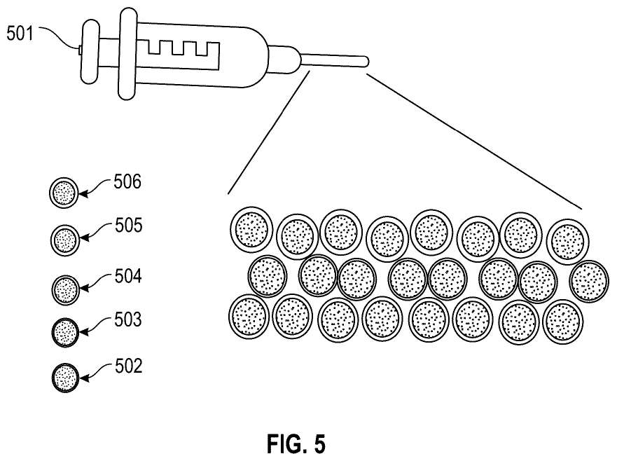

FIG. 5 shows an illustration of an exemplary procedure to induce cyclic surges in a medication level such as short acting immediate release LHRH. An injector 501 contain a species of short acting immediate release medication encapsulated in capsules, 502, 503, 504, 505 and 506. For example, when the thickness of the capsulating material for 502 is denoted as T mm, the thickness of the capsulating material for 503 may be 2T mm, the thickness of the capsulating material for 504 may be 3T mm, the thickness of the capsulating material for 505 may be 4T mm, and/or the thickness of the capsulating material for 506 may be 4.5T mm, allowing sequential release of the same medication.

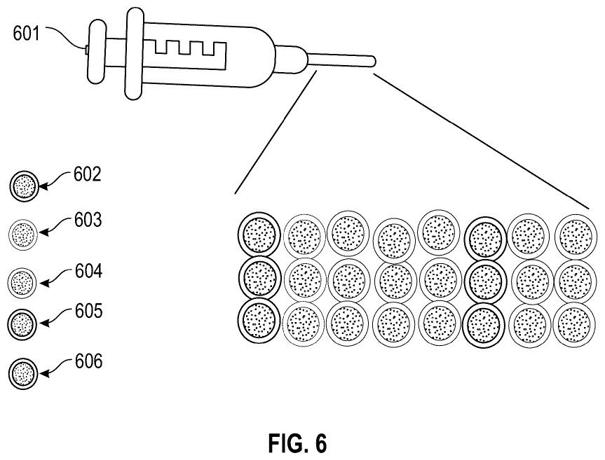

FIG. 6 shows an exemplary procedure to induce cyclic surges in a medication level such as short acting immediate release LHRH. An injection 601 contain a species of short acting immediate release medication encapsulated in material that has different densities (D mmol/lit) so that each density results in different dissolution time of the capsules and different release times of the medication inside the capsules. 1 D mmol/lit 602, 2 D mmol/lit 603, 3 D mmol/lit 604, 4 D mmol/lit 605, 5 D mmol/lit 606.

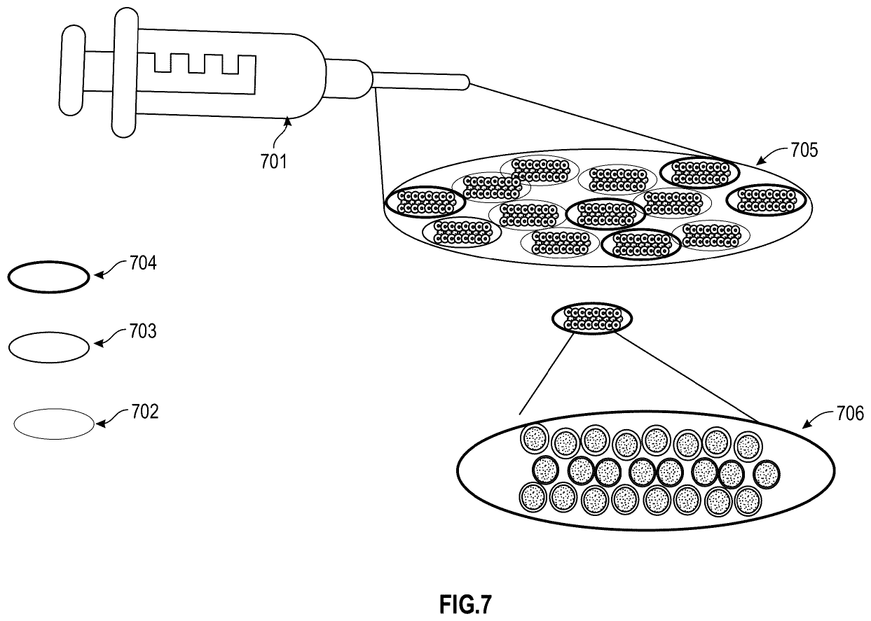

FIG. 7 shows the exemplary procedure with a needle 701 containing a plurality of master capsules 705, and each of the master capsules contains multiple species of capsules containing the same medication 706 that allows longer cyclic changes in the medication levels through variable temporal release of the medication. When the thickness of the capsules 702 is denoted as 1T mm, the thickness of the capsules 703 may be 2T mm, and the thickness of the capsules 704 may be 3T mm.

According to certain exemplary embodiments of the present disclosure, the following methods, compositions, products, kits and uses thereof can be provided:

Para. 1. A method of synthetic lethality for treatment of cancer, combining hormonal therapy and mitosis targeting treatments. Providing hormonal therapy or medications that induce secretion of one or more hormone that induces specific cancer cells to start mitosis, and provide in the same time or in proximity to that time treatments that kills cells in mitosis such as chemotherapy or radiation therapy.

Para. 2. A method to treat cancer in which the delivery of immediate release LHRH results in a surge in testosterone levels in men, and estrogen levels in women, driving respectively prostate and breast cancer cells, respectively, into mitosis, immediately before exposing them to mitosis targeting chemotherapy or radiation.

Para. 3. Immediate LHRH agonist, that is provided 4-120 hours before chemotherapy or radiation.

Para. 4. A method to increase serum endogenous estrogen for women to enhance estrogen secretion from the ovaries 8-120 hours before and during radiation, in estrogen-receptor-positive breast cancer patients, as a method for synthetic lethality during radiotherapy for ER+breast cancer.

Para. 5. A method to induce cyclic changes in the levels of GnRH agonist (or LHRH), to result in secretion of target hormones from the pituitary and subsequently from the gonads.

Para. 6. A method in any one of Paras. 1-5, wherein short acting LHRH agonist is used.

Para. 7. A method in any one of Paras. 1-6, in which LHRH is encapsulated in a variety of capsules formulations (n), that have different release times, so that formulation n+1 is released only after specific time form the release of LHRH from formulation n.

Para. 8. A method in any one of Paras. 1-7, in which the time between the release of LHRH from capsule n and n+1, is equal to z half-lives of LHRH.

Para. 9. A method in any one of Paras. 1-8, in which different formulations of LHRH with different half-lives are encapsulated in a same capsule X, or in different capsules A-Z, to achieve desired fluctuations in the levels of LHRH with time.

Para. 10. A method in any one of Paras. 1-9 to increase testosterone before therapies that induce mitosis targeted cell kill in patients with malignancies that has the androgen receptor.

Para. 11. A method in any one of Paras. 1-9 to increase estrogen before therapies that induce mitosis targeted cell kill in patients with malignancies that has estrogen or progesterone receptors.

Para. 12. A method in any one of Paras. 1-11, in which cell kill is done through radiation therapy, or chemotherapy.

Para. 13. A method in any one of Paras. 1-12, wherein chemotherapy include, but limited to, anthracyclines, plant Alkaloids, taxanes, vinca alkaloids; platinum-based chemotherapy, antimetabolites or Topoisomerase Inhibitors.

Para. 14. A method in any one of Paras. 1-13, to increase estrogen or testosterone blood levels before and at time of radiation therapy.

Para. 15. A method in any one of Paras. 1-14 in which luteinizing hormone releasing hormone agonist is provided in a way that it gets released in a cyclic manner into the blood or the extracellular matrix, to induce continuous release of luteinizing hormone from the pituitary and testosterone from the testicles, or estrogen from the ovaries.

Para. 16. A method in any one of Paras. 1-15 to increase serum endogenous testosterone to treat men with impotence. In this method short acting immediate release LHRH is delivered periodically to induce testosterone secretion from the gonads.

Para. 17. A method in any one of Paras. 1-16 to increase serum endogenous estrogen for women who need a surge in estrogen or progesterone, for example for in vitro fertilization.

Para. 18. A method in any one of Paras. 1-17 in which cyclic secretion of encapsulated said hormones or peptides is used to induce cyclic changes in said hormones, such as Corticotropin-releasing hormone (CRH) to activate the synthesis and release of adrenocorticotropic hormone (ACTH) from the pituitary gland when secreted in cyclic manner; thyrotropin-releasing hormone encapsulation that allows cyclic release of the hormones to induce thyroid activity; Growth hormone-releasing hormone cyclic secretion to induce the endogenous production of growth hormone, as alternative to treatment with growth hormone.

Para. 19. A method in any one of Paras. 1-18 to increase serum endogenous estrogen for women to enhance estrogen secretion from the ovaries as part of fertility treatment.

Para. 20. A method to deliver a peptide to the body in a cyclic manner, in which said peptide is encapsulated in a variety of capsules n, and each capsule releases the peptide at different time, and wherein said capsules are incorporated in transdermal patch.

Para. 21. A method to deliver a peptide in a cyclic manner, wherein the peptide is delivered through a pump in a programmed manner to the subcutaneous or intravascular space.

Para. 22. A method to deliver a peptide to the body in a cyclic manner, in which said peptide is encapsulated in a variety of capsules n, and each capsule releases the peptide at different time, and wherein said capsules are delivered through an intranasal route, including but not limited to spray, intranasal patch, syringe or other.

Para. 23. A method to increase endogenous hormonal secretion from the adrenal cortex by providing encapsulated pulsatile CRH, to induce cyclic changes in its levels, so the pituitary produces ACTH which acts on the adrenal glands to produce cortisol.

Para. 24. A method to provide cyclical Corticotropin-releasing hormone therapy for treatment of depression, anxiety and addiction.

Para. 25. A method to provide surge in Growth Hormone-Releasing Hormone in a cyclic manner to induce the endogenous production of growth hormone, as alternative to treatment with growth hormone.

Para. 26. A method to provide thyrotropin-releasing hormone encapsulation that allows cyclic release of the hormones to induce thyroid activity.

Para. 27. A drug delivery pellets 1−n, comprising: a core portion comprising a therapeutic selected from the group consisting of proteins, polypeptides, and combinations thereof; and a coating for the core portion, the coating and core portion forming a discrete body, the coating comprising a hydrophilic gel-forming agent undercoating or mixed with a polymeric rate-controlling material, wherein the release of the therapeutic from pellet x is different from the time of release of the therapeutic from pellet x+1.

Para. 28. Compactable self-scaling, drug delivery pellets, each pellet comprising: a core portion comprising an active agent selected from the group consisting of proteins, polypeptides, and combinations thereof; and a coating for the core portion, the coating and core portion forming a discrete body, the coating comprising a mixed layer of a hydrophilic gel-forming agent, a polymeric rate-controlling material, and additional coatings, wherein the coating of pellet x is different from the coating of pellet x+1, to allow phased delivery of the therapeutic for the different pellets families.

Para. 29. A self-injectable prefilled needle, that can be pen shaped, that had a prefilled syringe that is permanent or disposable, that include LHRH, LHRH agonist, decapeptide (pGlu-His-Trp-Ser-Tyr-D-Trp-Leu-Arg-Pro-Gly-NH₂) or any modification of this said peptide that maintain agonist effect on the pituitary to release LH.

Para. 30. The self-injectable prefilled needle according to Para. 29 in which the LHRH, LHRH agonist or decapeptide are in a liquid form.

Para. 31. The method according to any one of Paras. 29-30 in which the active material is stored in a protein preserving environment, which can include but not limited to zinc, m-cresol, glycerol, polysorbate 20, Lactose, monohydrate, magnesium stearate, methylhydroxypropylcellulose, polyethylene glycol, polyvidone, sodium starch glycollate, titanium dioxide and water for injection.

Para. 32. The method according to any one of Paras. 29-31, in which a cancer patient is prescribed the said decapeptide/LHRH pen. The patient self-injects a dose of the medicine (LHRH agonist immediate release) to himself 4-120 hours before the chemotherapy scheduled, wherein the chemotherapy is any material that target mitotic or dividing cells, including but not limited to taxanes (docetaxel, paclitaxel).

Para. 33. The method according to any one of Paras. 29-32 in which a premenopausal breast cancer patient with hormone receptor positive tumor, self-inject LHRH agonist immediate release medication to herself 4-120 hours before the chemotherapy is scheduled.

Para. 34. The method according to any one of Paras. 29-33 in which patients with hormone sensitive malignancy (prostate, breast, etc. . . . ) and who are schedule for radiation therapy targeting the tumor or microscopic tumor, self-inject LHRH agonist immediate release 4-48 hours before the start of the radiation week, so radiation is delivered when tumor cells are dividing.

Para. 35. The method according to Para. 34 in which the patient provides repeated injections to himself based on blood hormone level measurements.

Para. 36. The method according to any one of Paras. 29-35, in which the self-injectable pen is used to treat impotence, in which the patient self-injects immediate release LHRH every 1-21 days to increase endogenous testosterone levels.

Para. 37. A method to treat HER2 positive malignancies, such as breast and stomach cancer, in which the epitope that activates HER2 is provided intravenously, subcutaneously, or intramuscularly to the patient 4-120 hours before chemotherapy administration, so cancer cells get into mitosis, priming them to cell kill by chemotherapy.

Para. 38. The method according to Para. 37, where in the epitope that activates HER2 is provided in a vector that includes its DNA structure, wherein that said vector have an upstream control unit that can be activated by other medication such as antibiotic. Activation of the expression of the vector is done by providing said activating medication between 4-96 hours before chemotherapy to get tumor cells overexpressing HER2 to enter into the mitosis phase, priming them to cell kill by chemotherapy.

Para. 39. The method according to Para. 38 where in a tetracycline-based transcription regulation system is introduced upstream of the sequence for human epidermal growth factor, in retroviral, lentiviral vectors, rAAV and HSV vectors, for gene regulation. A method in which tetracycline is delivered before chemotherapy to further enhance the division of HER2 expressing cells, to enhance human-epidermal-growth-factor-expressing-cell kill by chemotherapy.

Para. 40. The method according to any one of Paras. 37-39 where an X-ray-radiation-induced promoters, are provided in vectors that express human epidermal growth factor sequence, so that during radiation, the breast tumor cells overexpress the human epidermal growth factor that activates the tumor cell division, and increase tumor cell kill by radiation.

Para. 41. The method according to any one of Paras. 37-40, in which the target receptor is Epidermal growth factor receptor (EGFR) and the epitope is Epidermal growth factor. So, overexpression or delivery of Epidermal growth factor is done before chemotherapy to increase cell division of lung cancer or head and neck tumors that overexpress EGFR to prime them for cell kill by chemotherapy or radiation.

Para. 42. The method according to any one of Paras. 37-41, in which a tumor that overexpress a specific growth receptor, in which the epitope that activates this said receptor is provided before chemotherapy or radiation to increase cell kill by chemotherapy or radiation.

Para. 43. The method according to any one of Paras. 29-33 in which patients with hormone sensitive malignancy (prostate, breast, etc.) and who are schedule for radiation or chemotherapy targeting the tumor, get an injection of the hormone to which the cancer cells have a receptor (such as testosterone or estrogen), 4-120 hours before the start of the radiation or the chemotherapy, so radiation or chemotherapy is delivered when tumor cells are dividing.

Para. 44. An antibody that targets EGFR and HER2 and results in its dimerization and activation, increases mitosis of cells that have these receptors. This antibody is given before radiation or chemotherapy.

Para. 45. A method to treat hormone receptor positive breast cancer, in premenopausal women, in which the timing of providing mitosis targeting chemotherapy is coordinated with the menstrual cycle so that chemotherapy is provided when the ovary is in the follicular phase of the menstrual cycle, when estradiol approaches peak levels, for Estrogen receptor positive cancer. Or providing mitosis targeting chemotherapy for estrogen receptor negative, progesterone receptor positive, breast cancer during the luteal phase of the ovarian cycle, when progesterone levels reach a peak levels.

Para. 46. A method to induce supraphysiologic levels of estrogen in premenopausal women by either the induction of maturation of multiple ovarian follicles, or by providing estrogen pills or injections.

Para. 47. A method to treat estrogen receptor positive breast cancer, in which estrogen is provided at high doses 24-72 hours before providing chemotherapy that targets mitotic cells.

Para. 48. A method to treat progesterone receptor positive breast cancer, in which progesterone is provided at high doses 24-72 hours before providing chemotherapy that targets mitotic cells.

Para. 49. A method to treat estrogen receptor positive/progesterone receptor positive breast cancer, in which estrogen and progesterone are provided at high doses 24-72 hours before providing chemotherapy that targets mitotic cells.

Para. 50. The method according to any one of Paras. 47-49 in which long acting LHRH agonist is delivered, or repeated daily injections of LHRH agonist such as leuprolide are delivered to suppress LH/FSH release from the hypophysis. Estrogen (estradiol), progesterone or both, are then delivered 12-72 hours before chemotherapy course that targets cell in mitosis, so cell kill is enhanced by the estrogen/progesterone delivered before the chemotherapy.

Para. 51. A method to treat hormone sensitive breast cancer in which estardiol or estrogen is provided to the patient in a self-injectable pen or single use needle, so the patient delivers the medication to herself 12-72 hours before the chemotherapy is scheduled.

Para. 52. A method to treat hormone sensitive breast cancer in which progesterone is provided to the patient in a self-injectable pen or single use needle, so the patient delivers the medication to herself 12-72 hours before the chemotherapy is scheduled.

Para. 53. A method to treat hormone sensitive breast cancer in which a combination of estardiol/estrogen and progesterone is provided to the patient in a self-injectable pen or single use needle, so the patient delivers the medication to herself 12-72 hours before the chemotherapy is scheduled.

Para. 54. The method according to any one of Paras. 51-53 in which premenopausal patient, is treated with long-acting continuous release LHRH agonist, provided before starting estrogen, progesterone or chemotherapy.

Para. 55. The method according to any one of Paras. 1-54 in which the chemotherapy in a continuous infusion pump, after induction of tumor cell mitosis by hormonal therapy, so the tumor cell-kill by chemotherapy span the period in which the tumor cells are in mitosis.

Para. 56. The method according to Para. 55, in which the pump is programed to deliver the hormonal therapy at specific time point first, and to deliver the chemotherapy after a specific amount of hours after that, as a push or continuous infusion.

Para. 57. The method according to any one of Paras. 51-56 in which treatment of hormone sensitive breast cancer is done by providing estrogen and/or progesterone in conjunction with enoxaparin or low molecular weight heparin (LMWH) to prevent hypercoagulability induced by hormonal therapy, followed by providing chemotherapy that target mitosis, while stopping the LMWH at or before day 5 or before the platelets levels is expected to arrive to nadir.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are incorporated herein by reference, in their entireties:
1. Huggins, C.; Hodges, C.V. Studies on prostatic cancer. Cancer Res 1941, 1, 293-297.
2. Saffran, M.; Schally, A.V. The release of corticotrophin by anterior pituitary tissue in vitro. Canadian journal of biochemistry and physiology 1955, 33, 408-415.
3. Matsuo, H.; Baba, Y.; Nair, R.G.; Arimura, A.; Schally, A. Structure of the porcine LH-and FSH-releasing hormone. I. The proposed amino acid sequence. Biochemical and biophysical research communications 1971, 43, 1334-1339.
4. Baba, Y.; Matsuo, H.; Schally, A.V. Structure of the porcine LH- and FSH-releasing hormone. II. Confirmation of the proposed structure by conventional sequential analyses. Biochemical and Biophysical Research Communications 1971, 44, 459-463, doi:https://doi.org/10.1016/0006-291X(71)90623-1.
5. Eisenberger, M.A.; Blumenstein, B.A.; Crawford, E.D.; Miller, G.; McLeod, D.G.; Loehrer, P.J.; Wilding, G.; Scars, K.; Culkin, D.J.; Thompson, I.M., et al. Bilateral Orchiectomy with or without Flutamide for Metastatic Prostate Cancer. New England Journal of Medicine 1998, 339, 1036-1042, doi:10.1056/nejm199810083391504.
6. Tree, A.; Griffin, C.; Syndikus, I.; Birtle, A.; Choudhury, A.; Graham, J.; Ferguson, C.; Khoo, V.; Malik, Z.; O'Sullivan, J., et al. Non-randomised comparison of efficacy and side effects of bicalutamide compared with LHRH analogues in com-bination with radiotherapy in the xxxxxx trial. International Journal of Radiation Oncology*Biology*Physics 2022, https://doi.org/10.1016/j.ijrobp.2021.12.160, doi:https://doi.org/10.1016/j.ijrobp.2021.12.160.
7. Bolla, M.; Gonzalez, D.; Warde, P.; Dubois, J.B.; Mirimanoff, R.-O.; Storme, G.; Bernier, J.; Kuten, A.; Sternberg, C.; Gil, T., et al. Improved Survival in Patients with Locally Advanced Prostate Cancer Treated with Radiotherapy and Goserelin. New England Journal of Medicine 1997, 337, 295-300, doi:10.1056/nejm199707313370502.
8. Leuprolide versus Diethylstilbestrol for Metastatic Prostate Cancer. New England Journal of Medicine 1984, 311, 1281-1286, doi:10.1056/nejm198411153112004.
9. FUJIWARA, M.; YUASA, T.; KOMAI, Y.; FUJIWARA, R.; OGUCHI, T.; NUMAO, N.; YAMAMOTO, S.; YONESE, J. Switch-ing Patients With Prostate Cancer from GnRH Antagonist to Long-acting LHRH Agonist for Androgen Deprivation: Reducing Hospital Visits During the Coronavirus Pandemic. Bone 2021, 22, 68.68.
10. Steinberg, M. Degarelix: A gonadotropin-releasing hormone antagonist for the management of prostate cancer. Clinical Therapeutics 2009, 31, 2312-2331, doi:https://doi.org/10.1016/j.clinthera.2009.11.009.
11. Dearnaley, D.P.; Saltzstein, D.R.; Sylvester, J.E.; Karsh, L.; Mehlhaff, B.A.; Pieczonka, C.; Bailen, J.L.; Shi, H.; Ye, Z.; Faes-sel, H.M., et al. The Oral Gonadotropin-releasing Hormone Receptor Antagonist Relugolix as Neoadjuvant/Adjuvant Androgen Deprivation Therapy to External Beam Radiotherapy in Patients with Localised Intermediate-risk Prostate Cancer: A Randomised, Open-label, Parallel-group Phase 2 Trial. European Urology 2020, 78, 184-192, doi:https://doi.org/10.1016/j.eururo.2020.03.001.
12. Asakawa, J.; Iguchi, T.; Tamada, S.; Yasuda, S.; Ninomiya, N.; Kato, M.; Yamasaki, T.; Ohmachi, T.; Nakatani, T. A change from gonadotropin releasing hormone antagonist to gonadotropin releasing hormone agonist therapy does not affect the oncological outcomes in hormone sensitive prostate cancer. Basic and clinical andrology 2018, 28, 1-7.
13. Shore, N.D.; Saad, F.; Cookson, M.S.; George, D.J.; Saltzstein, D.R.; Tutrone, R.; Akaza, H.; Bossi, A.; van Veenhuyzen, D.F.; Selby, B., et al. Oral Relugolix for Androgen-Deprivation Therapy in Advanced Prostate Cancer. New England Journal of Medicine 2020, 382, 2187-2196, doi:10.1056/NEJMoa2004325.
14. Warde, P.; Mason, M.; Ding, K.; Kirkbride, P.; Brundage, M.; Cowan, R.; Gospodarowicz, M.; Sanders, K.; Kostashuk, E.; Swanson, G., et al. Combined androgen deprivation therapy and radiation therapy for locally advanced prostate cancer: a randomised, phase 3 trial. The Lancet 2011, 378, 2104-2111, doi:https://doi.org/10.1016/S0140-6736(11)61095-7.
15. Van Poppel, H.; Klotz, L. Gonadotropin-releasing hormone: an update review of the antagonists versus agonists. Inter-national journal of urology: official journal of the Japanese Urological Association 2012, 19, 594-601, doi:10.1111/j.1442-2042.2012.02997.x.
16. Sciarra, A.; Busetto, G.M.; Salciccia, S.; Del Giudice, F.; Maggi, M.; Crocetto, F.; Ferro, M.; De Berardinis, E.; Scarpa, R.M.; Porpiglia, F., et al. Does Exist a Differential Impact of Degarelix Versus LHRH Agonists on Cardiovascular Safety? Evi-dences From Randomized and Real-World Studies. Frontiers in endocrinology 2021, 12, 695170, doi:10.3389/fendo.2021.695170.
17. Klotz, L.; Boccon-Gibod, L.; Shore, N.D.; Andreou, C.; Persson, B.-E.; Cantor, P.; Jensen, J.-K.; Olesen, T.K.; Schröder, F.H. The efficacy and safety of degarelix: a 12-month, comparative, randomized, open-label, parallel-group. BJU international 2008, 2.
18. Sasagawa, I.; Kubota, Y.; Nakada, T.; Suzuki, H.; Hirano, J.; Sugano, O.; Kato, H.; Imamura, A.; Mastushita, K.; Onmura, Y., et al. Influence of luteinizing hormone-releasing hormone analogues on serum levels of prostatic acid phosphatase and prostatic specific antigen in patients with metastatic carcinoma of the prostate. International urology and nephrology 1998, 30, 745-753, doi:10.1007/bf02564863.
19. Verhelst, J.; Denis, L.; Van Vliet, P.; Van Poppel, H.; Braeckman, J.; Van Cangh, P.; Mattelaer, J.; D'Hulster, O.; Mahler, C. Endocrine profiles during administration of the new non-steroidal anti-androgen Casodex in prostate cancer. Clinical endocrinology 1994, 41, 525-530.
20. Iversen, P.; Melezinek, I.; Schmidt, A. Nonsteroidal antiandrogens: a therapeutic option for patients with advanced prostate cancer who wish to retain sexual interest and function. BJU international (Papier) 2001, 87, 47-56.
21. McLEOD, D.G.; Iversen, P.; See, W.A.; Morris, T.; Armstrong, J.; Wirth, M.P.; Group, C.E.P.C.T. Bicalutamide 150 mg plus standard care vs standard care alone for early prostate cancer. BJU international 2006, 97, 247-254.
22. Oh, W.K.; Landrum, M.B.; Lamont, E.B.; McNeil, B.J.; Keating, N.L. Does oral antiandrogen use before leuteinizing hormone-releasing hormone therapy in patients with metastatic prostate cancer prevent clinical consequences of a testosterone flare? Urology 2010, 75, 642-647, doi:10.1016/j.urology.2009.08.008.
23. Vis, A.N.; van der Sluis, T.M.; Al-Itejawi, H.H.M.; van Moorselaar, R.J.A.; Meuleman, E.J.H. Risk of disease flare with LHRH agonist therapy in men with prostate cancer: myth or fact? Urologic oncology 2015, 33, 7-15, doi:10.1016/j.urolonc.2014.04.016.
24. Krakowsky, Y.; Morgentaler, A. Risk of Testosterone Flare in the Era of the Saturation Model: One More Historical Myth. European Urology Focus 2019, 5, 81-89, doi:https://doi.org/10.1016/j.euf.2017.06.008.
25. Shipley, W.U.; Seiferheld, W.; Lukka, H.R.; Major, P.P.; Heney, N.M.; Grignon, D.J.; Sartor, O.; Patel, M.P.; Bahary, J.-P.; Zietman, A.L., et al. Radiation with or without Antiandrogen Therapy in Recurrent Prostate Cancer. New England Journal of Medicine 2017, 376, 417-428, doi:10.1056/NEJMoa1607529.
26. Carrie, C.; Hasbini, A.; de Laroche, G.; Richaud, P.; Guerif, S.; Latorzeff, I.; Supiot, S.; Bosset, M.; Lagrange, J.-L.; Beckendorf, V., et al. Salvage radiotherapy with or without short-term hormone therapy for rising prostate-specific antigen concentration after radical prostatectomy (GETUG-AFU 16): a randomised, multicentre, open-label phase 3 trial. The Lancet Oncology 2016, 17, 747-756, doi:https://doi.org/10.1016/S1470-2045(16)00111-X.
27. Mout, L.; van Royen, M.E.; de Ridder, C.; Stuurman, D.; van de Geer, W.S.; Marques, R.; Buck, S.A.J.; French, P.J.; van de Werken, H.J.G.; Mathijssen, R.H.J., et al. Continued androgen signalling inhibition improves cabazitaxel efficacy in prostate cancer. EBioMedicine 2021, 73, 103681, doi:10.1016/j.ebiom.2021.103681.
28. Litvinov, I.V.; Vander Griend, D.J.; Antony, L.; Dalrymple, S.; De Marzo, A.M.; Drake, C.G.; Isaacs, J.T. Androgen receptor as a licensing factor for DNA replication in androgen-sensitive prostate cancer cells. Proceedings of the National Academy of Sciences of the United States of America 2006, 103, 15085-15090, doi:10.1073/pnas.0603057103.
29. Chute, R.; Willetts, A.T. The Treatment of Cancer of the Prostate with Castration and the Administration of Estrogen: A Preliminary Report. New England Journal of Medicine 1942, 227, 863-869.
30. Hussain, M.; Tangen, C.M.; Berry, D.L.; Higano, C.S.; Crawford, E.D.; Liu, G.; Wilding, G.; Prescott, S.; Kanaga Sundaram, S.; Small, E.J., et al. Intermittent versus Continuous Androgen Deprivation in Prostate Cancer. New England Journal of Medicine 2013, 368, 1314-1325, doi:10.1056/NEJMoa1212299.
31. Sharifi, N.; Gulley, J.L.; Dahut, W.L. Androgen deprivation therapy for prostate cancer. Jama 2005, 294, 238-244.
32. Kiwata, J.L.; Dorff, T.B.; Schroeder, E.T.; Gross, M.E.; Dieli-Conwright, C.M. A review of clinical effects associated with metabolic syndrome and exercise in prostate cancer patients. Prostate Cancer and Prostatic Diseases 2016, 19, 323-332, doi:10.1038/pcan.2016.25.
33. Nasser, N.J.; Sun, k.; Scanlon, K.M.; Mishra, M.V.; Molitoris, J.K. Administering Docetaxel for Metastatic Hormone Sensitive Prostate Cancer 1-6 Days Compared to More than 14 Days After the Start of LHRH Agonist is Associated with Better Clinical Outcomes Due to Androgen Flare. Cancers 2022, 14, 1-12, doi:doi.org/10.3390/cancers14040864.
34. Hernández-Vargas, H.; Palacios, J.; Moreno-Bueno, G. Telling cells how to die: docetaxel therapy in cancer cell lines. Cell Cycle 2007, 6, 780-783.
35. Pienta, K.J. Preclinical mechanisms of action of docetaxel and docetaxel combinations in prostate cancer. Seminars in Oncology 2001, 28, 3-7, doi:https://doi.org/10.1016/S0093-7754(01)90148-4.
36. Ashrafizadeh, M.; Mirzaei, S.; Hashemi, F.; Zarrabi, A.; Zabolian, A.; Saleki, H.; Sharifzadeh, S.O.; Soleymani, L.; Daneshi, S.; Hushmandi, K., et al. New insight towards development of paclitaxel and docetaxel resistance in cancer cells: EMT as a novel molecular mechanism and therapeutic possibilities. Biomedicine & Pharmacotherapy 2021, 141, 111824, doi:https://doi.org/10.1016/j.biopha.2021.111824.
37. Fabbri, F.; Amadori, D.; Carloni, S.; Brigliadori, G.; Tesei, A.; Ulivi, P.; Rosetti, M.; Vannini, I.; Arienti, C.; Zoli, W. Mi-totic catastrophe and apoptosis induced by docetaxel in hormone-refractory prostate cancer cells. Journal of cellular physiology 2008, 217, 494-501.
38. Gourdin, T.S.; Lilly, M.B.; Hussain, A.; Savage, S.; Clarke, H.S.; Sion, A.M.; Grubb, R.; Sellman, D.; Dincman, T.; Mikoll, J. Preliminary results from a phase II trial of docetaxel before castration with degarelix in men with newly diagnosed metastatic prostate cancer. American Society of Clinical Oncology: 2021.
39. Kramer, G.; Schwarz, S.; Hägg, M.; Havelka, A.M.; Linder, S. Docetaxel induces apoptosis in hormone refractory prostate carcinomas during multiple treatment cycles. British journal of cancer 2006, 94, 1592-1598.
40. Mohammadian, J.; Sabzichi, M.; Molavi, O.; Shanehbandi, D.; Samadi, N. Combined treatment with stattic and docetaxel alters the Bax/Bcl-2 gene expression ratio in human prostate cancer cells. Asian Pacific journal of cancer prevention: APJCP 2016, 17, 5031.
41. Sweeney, C.J.; Chen, Y.-H.; Carducci, M.; Liu, G.; Jarrard, D.F.; Eisenberger, M.; Wong, Y.-N.; Hahn, N.; Kohli, M.; Cooney, M.M. Chemohormonal therapy in metastatic hormone-sensitive prostate cancer. New England Journal of Medi-cine 2015, 373, 737-746.
42. Clarke, N.W.; Ali, A.; Ingleby, F.C.; Hoyle, A.; Amos, C.L.; Attard, G.; Brawley, C.D.; Calvert, J.; Chowdhury, S.; Cook, A., et al. Addition of docetaxel to hormonal therapy in low- and high-burden metastatic hormone sensitive prostate cancer: long-term survival results from the STAMPEDE trial. Annals of Oncology 2019, 30, 1992-2003, doi:https://doi.org/10.1093/annonc/mdz396.
43. James, N.D.; Sydes, M.R.; Clarke, N.W.; Mason, M.D.; Dearnaley, D.P.; Spears, M.R.; Ritchie, A.W.; Parker, C.C.; Russell, J.M.; Attard, G. Addition of docetaxel, zoledronic acid, or both to first-line long-term hormone therapy in prostate cancer (STAMPEDE): survival results from an adaptive, multiarm, multistage, platform randomised controlled trial. The Lancet 2016, 387, 1163-1177.
44. Kyriakopoulos, C.E.; Chen, Y.-H.; Carducci, M.A.; Liu, G.; Jarrard, D.F.; Hahn, N.M.; Shevrin, D.H.; Dreicer, R.; Hussain, M.; Eisenberger, M., et al. Chemohormonal Therapy in Metastatic Hormone-Sensitive Prostate Cancer: Long-Term Survival Analysis of the Randomized Phase III E3805 CHAARTED Trial. Journal of clinical oncology: official journal of the American Society of Clinical Oncology 2018, 36, 1080-1087, doi:10.1200/JCO.2017.75.3657.
45. Hall, E.J.; Giaccia, A.J. Radiobiology for the Radiologist, Eight Edition ed.; Philadelphia: 2019.
46. Bolla, M.; Van Tienhoven, G.; Warde, P.; Dubois, J.B.; Mirimanoff, R.-O.; Storme, G.; Bernier, J.; Kuten, A.; Sternberg, C.; Billiet, I. External irradiation with or without long-term androgen suppression for prostate cancer with high metastatic risk: 10-year results of an EORTC randomised study. The lancet oncology 2010, 11, 1066-1073.
47. Lawton, C.A.; Winter, K.; Grignon, D.; Pilepich, M.V. Androgen suppression plus radiation versus radiation alone for patients with stage D1/pathologic node-positive adenocarcinoma of the prostate: updated results based on national prospective randomized trial Radiation Therapy Oncology Group 85-31. Journal of Clinical Oncology 2005, 23, 800-807.
48. Parker, C.C.; James, N.D.; Brawley, C.D.; Clarke, N.W.; Hoyle, A.P.; Ali, A.; Ritchie, A.W.; Attard, G.; Chowdhury, S.; Cross, W. Radiotherapy to the primary tumour for newly diagnosed, metastatic prostate cancer (STAMPEDE): a ran-domised controlled phase 3 trial. The Lancet 2018, 392, 2353-2366.
49. van Leeuwen, C.M.; Oei, A.L.; Crezee, J.; Bel, A.; Franken, N.A.P.; Stalpers, L.J.A.; Kok, H.P. The alfa and beta of tumours: a review of parameters of the linear-quadratic model, derived from clinical radiotherapy studies. Radiation Oncology 2018, 13, 96, doi:10.1186/s13014-018-1040-z.
50. Hegemann, N.-S.; Guckenberger, M.; Belka, C.; Ganswindt, U.; Manapov, F.; Li, M. Hypofractionated radiotherapy for prostate cancer. Radiation Oncology 2014, 9, 275, doi:10.1186/s13014-014-0275-6.
51. Wilder, R.B.; Tucker, S.L.; Ha, C.S.; Rodriguez, M.A.; Hess, M.A.; Cabanillas, F.F.; Cox, J.D. Dose-response analysis for radiotherapy delivered to patients with intermediate-grade and large-cell immunoblastic lymphomas that have completely responded to CHOP-based induction chemotherapy. International journal of radiation oncology, biology, physics 2001, 49, 17-22, doi:10.1016/s0360-3016(00)01383-3.
52. Klement, R.J.; Sonke, J.J.; Allgäuer, M.; Andratschke, N.; Appold, S.; Belderbos, J.; Belka, C.; Dieckmann, K.; Eich, H.T.; Flentje, M., et al. Estimation of the α/β ratio of non-small cell lung cancer treated with stereotactic body radiotherapy. Radiotherapy and oncology: journal of the European Society for Therapeutic Radiology and Oncology 2020, 142, 210-216, doi:10.1016/j.radonc.2019.07.008.
53. Wang, J.Z.; Guerrero, M.; Li, X.A. How low is the alpha/beta ratio for prostate cancer? International journal of radiation oncology, biology, physics 2003, 55, 194-203, doi:10.1016/s0360-3016(02)03828-2.
54. Vogelius, I.R.; Bentzen, S.M. Meta-analysis of the Alpha/Beta Ratio for Prostate Cancer in the Presence of an Overall Time Factor: Bad News, Good News, or No News? International Journal of Radiation Oncology*Biology*Physics 2013, 85, 89-94, doi:https://doi.org/10.1016/j.ijrobp.2012.03.004.
55. Leborgne, F.; Fowler, J.; Leborgne, J.H.; Mezzera, J. Later Outcomes and Alpha/Beta Estimate From Hypofractionated Conformal Three-Dimensional Radiotherapy Versus Standard Fractionation for Localized Prostate Cancer. International Journal of Radiation Oncology*Biology*Physics 2012, 82, 1200-1207, doi:https://doi.org/10.1016/j.ijrobp.2010.12.040.
56. Deutsch, I.; Zelefsky, M.J.; Zhang, Z.; Mo, Q.; Zaider, M.; Cohen, G.a.; Cahlon, O.; Yamada, Y. Comparison of PSA relapse-free survival in patients treated with ultra-high-dose IMRT versus combination HDR brachytherapy and IMRT. Brachytherapy 2010, 9, 313-318, doi:https://doi.org/10.1016/j.brachy.2010.02.196.
57. Pilepich, M.V.; Winter, K.; Lawton, C.A.; Krisch, R.E.; Wolkov, H.B.; Movsas, B.; Hug, E.B.; Asbell, S.O.; Grignon, D. An-drogen suppression adjuvant to definitive radiotherapy in prostate carcinoma—long-term results of phase III RTOG 85-31. International journal of radiation oncology, biology, physics 2005, 61, 1285-1290, doi:10.1016/j.ijrobp.2004.08.047.
58. Roach, M.; Moughan, J.; Lawton, C.A.F.; Dicker, A.P.; Zeitzer, K.L.; Gore, E.M.; Kwok, Y.; Seider, M.J.; Hsu, I.C.; Hartford, A.C., et al. Sequence of hormonal therapy and radiotherapy field size in unfavourable, localised prostate cancer (NRG/RTOG 9413): long-term results of a randomised, phase 3 trial. The Lancet Oncology 2018, 19, 1504-1515, doi:https://doi.org/10.1016/S1470-2045(18)30528-X.
59. Bolla, M.; de Reijke, T.M.; Van Tienhoven, G.; Van den Bergh, A.C.M.; Oddens, J.; Poortmans, P.M.P.; Gez, E.; Kil, P.; Akdas, A.; Soete, G., et al. Duration of Androgen Suppression in the Treatment of Prostate Cancer. New England Journal of Medicine 2009, 360, 2516-2527, doi:10.1056/NEJMoa0810095.
60. Roach III, M.; Bae, K.; Speight, J.; Wolkov, H.B.; Rubin, P.; Lee, R.J.; Lawton, C.; Valicenti, R.; Grignon, D.; Pilepich, M.V. Short-term neoadjuvant androgen deprivation therapy and external-beam radiotherapy for locally advanced prostate cancer: long-term results of RTOG 8610. Journal of Clinical Oncology 2008, 26, 585-591.
61. Hanks, G.E.; Pajak, T.F.; Porter, A.; Grignon, D.; Brereton, H.; Venkatesan, V.; Horwitz, E.M.; Lawton, C.; Rosenthal, S.A.; Sandler, H.M., et al. Phase III Trial of Long-Term Adjuvant Androgen Deprivation After Neoadjuvant Hormonal Cytoreduction and Radiotherapy in Locally Advanced Carcinoma of the Prostate: The Radiation Therapy Oncology Group Protocol 92-02. Journal of Clinical Oncology 2003, 21, 3972-3978, doi:10.1200/jco.2003.11.023.
62. Lawton, C.A.; DeSilvio, M.; Roach, M., 3rd; Uhl, V.; Kirsch, R.; Seider, M.; Rotman, M.; Jones, C.; Asbell, S.; Valicenti, R., et al. An update of the phase III trial comparing whole pelvic to prostate only radiotherapy and neoadjuvant to adjuvant total androgen suppression: updated analysis of RTOG 94-13, with emphasis on unexpected hormone/radiation interac-tions. International journal of radiation oncology, biology, physics 2007, 69, 646-655, doi:10.1016/j.ijrobp.2007.04.003.
63. Kishan, A.U.; Sun, Y.; Hartman, H.; Pisansky, T.M.; Bolla, M.; Neven, A.; Steigler, A.; Denham, J.W.; Feng, F.Y.; Zapatero, A., et al. Androgen deprivation therapy use and duration with definitive radiotherapy for localised prostate cancer: an individual patient data meta-analysis. The Lancet Oncology 2022, 23, 304-316, doi:https://doi.org/10.1016/S1470-2045(21)00705-1.
64. Polkinghorn, W.R.; Parker, J.S.; Lee, M.X.; Kass, E.M.; Spratt, D.E.; Iaquinta, P.J.; Arora, V.K.; Yen, W.-F.; Cai, L.; Zheng, D., et al. Androgen Receptor Signaling Regulates DNA Repair in Prostate Cancers. Cancer Discovery 2013, 3, 1245-1253, doi:10.1158/2159-8290.cd-13-0172.
65. Morgentaler, A.; Traish, A.M. Shifting the Paradigm of Testosterone and Prostate Cancer: The Saturation Model and the Limits of Androgen-Dependent Growth. European Urology 2009, 55, 310-321, doi:https://doi.org/10.1016/j.eururo.2008.09.024.
66. Seidman, S.N.; Walsh, B.T. Testosterone and Depression in Aging Men. The American Journal of Geriatric Psychiatry 1999, 7, 18-33, doi:https://doi.org/10.1097/00019442-199902000-00004.
67. Yeap, B.B. Testosterone and ill-health in aging men. Nature Clinical Practice Endocrinology & Metabolism 2009, 5, 113-121, doi:10.1038/nependmet1050.
68. Klap, J.; Schmid, M.; Loughlin, K.R. The Relationship between Total Testosterone Levels and Prostate Cancer: A Review of the Continuing Controversy. The Journal of urology 2015, 193, 403-414, doi:https://doi.org/10.1016/j.juro.2014.07.123.
69. Schweizer, M.T.; Antonarakis, E.S.; Wang, H.; Ajiboye, A.S.; Spitz, A.; Cao, H.; Luo, J.; Haffner, M.C.; Yegnasubramanian, S.; Carducci, M.A., et al. Effect of bipolar androgen therapy for asymptomatic men with castration-resistant prostate cancer: results from a pilot clinical study. Science translational medicine 2015, 7, 269ra262, doi:10.1126/scitranslmed.3010563.
70. Mohammad, O.S.; Nyquist, M.D.; Schweizer, M.T.; Balk, S.P.; Corey, E.; Plymate, S.; Nelson, P.S.; Mostaghel, E.A. Su-praphysiologic Testosterone Therapy in the Treatment of Prostate Cancer: Models, Mechanisms and Questions. Cancers 2017, 9, 166.
71. Kumar, R.; Mendonca, J.; Owoyemi, O.; Boyapati, K.; Thomas, N.; Kanacharoen, S.; Coffey, M.; Topiwala, D.; Gomes, C.; Ozbek, B. Supraphysiologic Testosterone Induces Ferroptosis and Activates Immune Pathways through Nucleophagy in Prostate Cancer. Cancer Research 2021, 81, 5948-5962.
72. Nordeen, S.K.; Su, L.-J.; Osborne, G.A.; Hayman, P.M.; Orlicky, D.J.; Wessells, V.M.; van Bokhoven, A.; Flaig, T.W. Titration of Androgen Signaling: How Basic Studies Have Informed Clinical Trials Using High-Dose Testosterone Therapy in Castrate-Resistant Prostate Cancer. Life 2021, 11, 884.
73. Isaacs, J.T.; Brennen, W.N.; Denmeade, S.R. Serial bipolar androgen therapy (sBAT) using cyclic supraphysiologic testosterone (STP) to treat metastatic castration-resistant prostate cancer (mCRPC). Annals of translational medicine 2019, 7, S311, doi:10.21037/atm.2019.10.32.
74. Lam, H.-M.; Nguyen, H.M.; Labrecque, M.P.; Brown, L.G.; Coleman, I.M.; Gulati, R.; Lakely, B.; Sondheim, D.; Chatterjee, P.; Marck, B.T., et al. Durable Response of Enzalutamide-resistant Prostate Cancer to Supraphysiological Testosterone Is Associated with a Multifaceted Growth Suppression and Impaired DNA Damage Response Transcriptomic Program in Patient-derived Xenografts. European Urology 2020, 77, 144-155, doi:https://doi.org/10.1016/j.eururo.2019.05.042.
75. Denmeade, S.; Antonarakis, E.S.; Markowski, M.C. Bipolar androgen therapy (BAT): A patient's guide. Prostate 2022, 10.1002/pros.24328, doi:10.1002/pros.24328.
76. Hoshi, S.; Bilim, V.; Hoshi, K.; Nakagawa, T.; Sato, S.; Sakagami, R.; Konno, M.; Kudo, T.; Numahata, K.; Sasagawa, I. Clinical response in metastatic castration-resistant prostate cancer (mCRPC) cases treated with supra-physiological doses of testosterone: Bipolar androgen therapy. Clinical case reports 2022, 10, e05433, doi:10.1002/ccr3.5433.
77. Sena, L.A.; Wang, H.; Lim ScM, S.J.; Rifkind, I.; Ngomba, N.; Isaacs, J.T.; Luo, J.; Pratz, C.; Sinibaldi, V.; Carducci, M.A., et al. Bipolar androgen therapy sensitizes castration-resistant prostate cancer to subsequent androgen receptor ablative therapy. European Journal of Cancer 2021, 144, 302-309, doi:https://doi.org/10.1016/j.ejca.2020.11.043.
78. Mirzakhani, K.; Kallenbach, J.; Rasa, S.M.M.; Ribaudo, F.; Ungelenk, M.; Ehsani, M.; Gong, W.; Gassler, N.; Leeder, M.; Grimm, M.O., et al. The androgen receptor-lncRNASAT1-AKT-p15 axis mediates androgen-induced cellular senescence in prostate cancer cells. Oncogene 2022, 41, 943-959, doi:10.1038/s41388-021-02060-5.
79. Milosevic, M.; Chung, P.; Panzarella, T.; Toi, A.; Bristow, R.; Warde, P.; Catton, C.; Ménard, C.; Gospodarowicz, M.; Hill, R. 21: Androgen Deprivation Reduces Hypoxia in Human Prostate Cancer. International journal of radiation oncology, biology, physics 2006, 66, S12.
80. Baker, S.D.; Zhao, M.; Lee, C.K.K.; Verweij, J.; Zabelina, Y.; Brahmer, J.R.; Wolff, A.C.; Sparreboom, A.; Carducci, M.A. Comparative Pharmacokinetics of Weekly and Every-Three-Weeks Docetaxel. Clinical Cancer Research 2004, 10, 1976-1983, doi:10.1158/1078-0432.ccr-0842-03.
81. Nasser, N.J. Androgen Flare after LHRH Initiation Is the Side Effect That Makes Most of the Beneficial Effect When It Coincides with Radiation Therapy for Prostate Cancer. Cancers 14.8 2022, 1959.
82. Adjei, A., Sundberg, D., Miller, J. et al. Bioavailability of Leuprolide Acetate Following Nasal and Inhalation Delivery to Rats and Healthy Humans. Pharm Res 9, 244-249 (1992).
83. Kishan, A.U., et al. “Androgen deprivation therapy use and duration with definitive radiotherapy for localised prostate cancer: an individual patient data meta-analysis.” The Lancet Oncology 23.2 (2022): 304-316.
83. Tolis, G., et al. Tumor growth inhibition in patients with prostatic carcinoma treated with luteinizing hormone-releasing hormone agonists. Proceedings of the National Academy of Sciences 79.5 (1982): 1658-1662.
84. Fowler Jr, J. E., & Whitmore Jr, W. F. (1982). Considerations for the use of testosterone with systemic chemotherapy in prostatic cancer. Cancer, 49(7), 1373-1377.

1. A method for treating cancer in a subject, comprising:
(a) administering to the subject a medication in an amount effective to specifically induces mitosis of cancer cells, and
(b) applying a treatment that kills cells in mitosis 8-150 hours, 12-150 hours, or 8-120 hours after step (a).

2. The method according to claim 1, wherein the cancer is prostate cancer, and wherein the method comprises:
administering to the subject an immediate release LHRH agonist, such as Gonadorelin or a short acting Leuprolide, to induce an androgen surge without an ensuing androgen deprivation so that cells having an androgen receptor proceed to mitosis, and
administering to the subject, 8-120 hours after administering the immediate release LHRH agonist, a treatment that kills the cells in mitosis.

3. The method according to claim 1, wherein the cancer is hormone receptor positive breast cancer, wherein the subject is a premenopausal woman, and wherein the method further comprises:
administering to the subject an LHRH agonist at a dose of 0.1-1 mg/d starting day 21 of the menstrual cycle, to suppress the effect of the pituitary axis,
administering to the subject, at day 7 of the next cycle, intravenous estrogen (0.001-5 mg) to estrogen receptor positive breast cancer patients, or administering intravenous progesterone to progesterone receptor positive breast cancer, to specifically increase the part of the cells in mitosis, followed by the treatment 8-120 hours thereafter.

4. The method according to claim 1, wherein cancer is hormone receptor positive breast cancer, wherein the subject is a premenopausal woman, and wherein the method further comprises at least one of:
administering to the subject LHRH agonist at a dose of 0.1-1 mg/d starting day 21 of a menstrual cycle, to suppress the effect of the pituitary axis,
at day 1 of the next menstrual cycle, initiating an injection of follicle stimulating hormone (FSH) injections once daily for 8 days, at days 8-14, administering human chorionic gonadotropin (HCG) or LH, and at days 8-16, administering chemotherapy for estrogen receptor positive breast cancer, or
at days 12-20, administering chemotherapy for progesterone receptor positive breast cancer.

5. The method according to claim 4, wherein the chemotherapy is administered about 20-120 hours from a peak estrogen level or a peak progesterone level.

6. The method according to claim 1, wherein the cancer is a hormone receptor positive breast cancer, wherein the treatment includes a chemotherapy or a radiation therapy, and wherein the method further comprises administering to the subject estrogen or progesterone, or an analogue thereof, in an amount effective to increase a portion of the cancer cells in mitosis 8-120 hours before each cycle of the chemotherapy or the radiation therapy.

7. The method according to claim 1, wherein the cancer is human epidermal receptor 2 positive breast cancer, wherein the method further comprises administering a molecule that specifically activates a human epidermal growth factor receptor 8-120 hours before applying the treatment in step (b).

8. The method according to claim 1, wherein the medication in step (a) comprises a hormone or an analogue of the hormone.

9. The method according to claim 1, wherein the medications that induce tumor cell mitosis is administered intravenously, subcutaneously, transdermally, transbuccally, through an inhaler, by intranasal spray, or by a long intranasal applicator reaching as close as possible to the pituitary gland.

10. The method according to claim 1, wherein LHRH is encapsulated in a plurality of capsules formulations that have different release times, so that a formulation n+1 is released only after a specific time from a release of LHRH from a formulation n, resulting in a cyclic secretion of LHRH, with a peak between secretions ranging between 2 hours to 7 days, to achieve continuously higher than normal levels of testosterone.

11. A method for treating impotence in a subject with a low testosterone level, comprising:
administering to the subject short acting, immediate release LHRH via a self-delivery, pen shaped, subcutaneous needle injection, intranasal spray, trans buccal formulation, or through inhalers once every 1-4 weeks, or
administering to the subject an LHRH composition as described in claim 10, once every 1-6 months.

12. The method according to claim 1, wherein the chemotherapy includes using at least one of an anthracycline, a plant alkaloid, a taxane, a vinca alkaloid; a platinum-based chemotherapy, an antimetabolite or a topoisomerase inhibitor, or a combination thereof.

13. A kit comprising:
a self-injectable prefilled needle, comprising a prefilled syringe that is permanent or disposable, that includes an active agent comprising LHRH, an LHRH agonist, a decapeptide (pGlu-His-Trp-Ser-Tyr-D-Trp-Leu-Arg-Pro-Gly-NH2; SEQ ID NO:1), Gonadorelin, leuprolide, a modification thereof, or a combination thereof, and
an instruction for administering to a subject the active agent 8-150 hours before starting chemotherapy.

14. A method for treating a hormone sensitive malignancy in a subject, the method comprising:
administering to the subject at least one of:
a) an immediate release LHRH agonist or testosterone for treating prostate cancer 8-48 hours before a start of a radiation week so a radiation is delivered when tumor cells are dividing; or
b) progesterone for treating breast cancer 8-48 hours before the start of the radiation week, so that the radiation is delivered when the tumor cells are dividing.

15. A method for treating hormone receptor positive breast cancer, in a premenopausal subject, comprising:
providing a mitosis-targeting chemotherapy at least one of:
a) when at least one of (i) an ovary of the subject is in a follicular phase of a menstrual cycle or (ii) estradiol approaches peak levels for estrogen receptor positive cancer; or
b) for at least one of estrogen receptor negative, progesterone receptor positive, breast cancer during a luteal phase of an ovarian cycle of the subject, or when progesterone reaches a peak level.

16. A method according to claim 1, wherein the chemotherapy is administered by a continuous infusion pump, and wherein a tumor-cell-kill by the chemotherapy spans a period in which the tumor cells are in mitosis.

17. A method comprising delivering a therapeutic agent targeting the pituitary gland, alone or together with a pharmaceutically acceptable carrier through an intranasal device, wherein the therapeutic agent includes at least one of Gonadorelin, leuprolide, a Luteinizing Hormone-Releasing Hormone analogue, a Corticotropin-releasing hormone, a thyrotropin-releasing hormone, or a Growth hormone-releasing hormone.

18. The method according to claim 1, wherein the medication that specifically induces mitosis of cancer cells is gonadorelin, wherein the treatment includes a chemotherapy or a radiation therapy, and wherein 10 mcg-100 mcg gonadorelin is administered intravenously or subcutaneously 8-120 hours before the chemotherapy or the radiation therapy.

19. The method according to claim 17, wherein the therapeutic agent is delivered within a carrier that allows passing through bone.

20. The method according to claim 13, wherein the self-injectable prefilled needle is pen-shaped.

21. The method according to claim 8, wherein the hormone or the analogue of the hormone comprises at least one of testosterone, estrogen, progesterone, leuprolide, Gonadorelin, a Luteinizing Hormone-Releasing Hormone analogue, q Corticotropin-releasing hormone, a thyrotropin-releasing hormone, or a Growth hormone-releasing hormone.

22. The method according to claim 16, wherein the period in which the tumor cells are in mitosis are 8-150 hours after an induction of tumor cell mitosis.
